(19) United States
(12) Patent Application Publication
(10) Pub. No.: US 2020/0383885 A1
(43) Pub. Date: Dec. 10, 2020

(54) COSMETIC COMPOSITION COMPRISING LIQUID-PHASE PLASMA CAPABLE OF BEING STORED FOR LONG PERIOD OF TIME AS ACTIVE INGREDIENT FOR SKIN REGENERATION OR WHITENING

(71) Applicant: AJOU UNIVERSITY INDUSTRY-ACADEMIC COOPERATION FOUNDATION, Suwon-si, Gyeonggi-do (KR)

(72) Inventors: Jong Soo LEE, Seoul (KR); Sang Sik YANG, Seoul (KR)

(73) Assignee: AJOU UNIVERSITY INDUSTRY-ACADEMIC COOPERATION FOUNDATION, Suwon-si, Gyeonggi-do (KR)

(21) Appl. No.: 16/324,400

(22) PCT Filed: Aug. 11, 2017
(86) PCT No.: PCT/KR2017/008772

§ 371 Date: Apr. 22, 2019

(30) Foreign Application Priority Data
Aug. 11, 2016 (KR) 10-2016-0102285

(51) Int. Cl. A61K 8/19; A61Q 19/02; A61K 33/00; A61P 17/02; A61P 35/00; A61L 27/02; A23L 33/00; A23L 29/00; A61K 9/00

(52) U.S. Cl. CPC A61K 8/19; A61Q 19/02; A61K 33/00; A61P 17/02; A61K 2800/83; A61L 27/025; A23L 33/30; A23L 29/015; A61K 9/0053; A61P 35/00

(57) ABSTRACT

A composition including long-term storable plasma-activated liquid (liquid plasma) as an active ingredient is disclosed. The long-term storable, atmospheric-pressure, plasma-activated liquid is prepared by dissolving a plasma generated using a low-temperature microplasma jet in a solution. The plasma-activated liquid not only exhibits a cancer cell killing effect at a level similar to gas-phase plasma according to a conventional art, but also maintains the cancer cell killing effect even when the plasma-activated liquid is stored in a freezer or in a cold chamber for 6 months, and thus is suitable for long-term storage. The plasma-activated liquid may be effectively used in the biopharmaceutical field, for example for a skin regeneration, wound-healing of dermal cells, and/or treating a cancer.

This application is a national stage entry application of PCT/KR2017/008772 filed Aug. 11, 2017, which claims priority to and the benefit of Korean Patent Application No. 10-2016-0102285, filed on Aug. 11, 2016, the disclosures of which are incorporated herein by reference in their entirety.

### TECHNICAL FIELD

The present invention relates to a skin regeneration or whitening composition, which includes long-term storable plasma-activated liquid (liquid plasma) as an active ingredient; a composition for healing wounds; and a composition for preventing and treating cancer.

### BACKGROUND ART

Plasma is an ionized state of a gas, and generally includes electron ions and various radicals at a high density. Plasma has been reported to have properties of a fourth phase of matter, distinct from each of a solid, a liquid and a gas, and the first plasma preparing method was known to form a gas state in which negatively-charged electrons and a positively-charged ions are separated at a very high temperature. However, recently, to use plasma in biopharmaceutical applications, various methods capable of forming low-temperature, atmospheric-pressure plasma have been studied and reported. Among these methods, dielectric-barrier discharge is characterized by a high concentration of electron density and superior insulation, and has been widely used in the art to produce plasma.

As an application method capable of utilizing plasma in the biopharmaceutical field, studies on utilizing plasma in blood coagulation and wound healing are progressing. In addition, methods of utilizing plasma for selectively removing cancer cells have been reported, and therefore, a study on the possibility of using plasma for anticancer treatment was suggested (Non-Patent Literature 1). In addition, it has been reported, although the death of cancer cells can be induced by plasma, the risk of plasma with respect to human and normal cells is low, and thus studies have continued to develop application methods for generating low-temperature, atmospheric-pressure plasma for use in therapeutic applications (Non-Patent Literature 2, Non-Patent Literature 3, Patent Literature 1, and Patent Literature 2).

Cancer is one of the representative diseases with high mortality worldwide because recurrence or metastasis may occur after initial treatment, and the discovery and treatment of cancer itself may be delayed. In addition, since effective therapeutic methods such as one or more therapeutic methods of surgical treatment, radiation therapy and/or chemotherapy are different for each type of cancer, and the incidence of cancer has persistent internal heterogeneity of cancer, it is difficult to effectively cure cancer. Particularly, since treatment sensitivity varies according to a site where cancer occurs, even if the same treatment method is used, the recurrence and metastasis of cancer may occur, resulting in an increased risk of death.

Among the methods, in the area of an anticancer treatment using plasma, the inventors have reported, in a previous research, that various characteristics of plasma can be used to induce apoptosis of cancer cells due to DNA damage, mitochondrial collapse and the aberration of a cell membrane. In addition, it has been confirmed that active oxygen species (ROS) and active nitrogen species (RNS), which are generated by atmospheric-pressure plasma, can play a pivotal role in induction of cancer cell death (Non-Patent Literatures 2 to 5).

However, in application to the biopharmaceutical field, gaseous plasma has two significant limitations: it is practically impossible to transmit a therapeutic effect through tissue penetration, and long-term storage is difficult since it is gaseous. In other words, even though plasma generated at a low temperature and an atmospheric pressure has an excellent cancer cell killing effect, to be practically used as a therapeutic method and/or a drug, it is essential to prepare plasma in a form that has an excellent in-vivo delivery efficiency and suitability for long-term storage.

Therefore, Korean Patent No. 10-1001477 discloses a method of manufacturing an atmospheric-pressure and low-temperature microplasma jet to be used in a bio-medical application, and Korean Patent No. 10-1409390 discloses a method of killing disease-associated cells and pathogenic microorganisms using the atmospheric-pressure and low-temperature microplasma jet. In Korean Patent No. 10-1409390, it is shown that pathogenic microorganisms can be effectively killed by applying plasma to a solution such as a buffer solution or water and exposing a subject such as microorganisms or animal or plant cells to the solution for treatment. However, it has not been reported that a dermal wound healing effect or a cancer cell killing effect, in addition to the death of pathogenic microorganisms, can be exhibited using the plasma-activated liquid prepared as described above.

As a result of efforts made to apply plasma-activated liquid to the biopharmaceutical field, the inventors found that plasma-activated liquid prepared by dissolving plasma generated using an atmospheric-pressure and low-temperature microplasma jet in a solution not only exhibits a cancer cell killing effect at a level similar to gas-phase plasma in the conventional art, but also exhibits suitability for long-term storage since the cancer cell killing effect can be maintained even when the plasma-activated liquid is frozen for a long time. In addition, it was confirmed that the plasma-activated liquid of the present invention exhibits a regeneration effect on wound-induced dermal cells, and the plasma-activated liquid stored in a cold chamber or at room temperature for 6 months also exhibits a significant skin regeneration effect, demonstrating that the plasma-activated liquid of the present invention is suitable for long-term storage and thus can be used as an active ingredient for a composition for treating cancer or a cosmetic composition for skin regeneration. Therefore, the present invention was completed.

### PRIOR ART LITERATURES

### Patent Literatures

(Patent literature 1) KR 10-1001477 B1
(Patent literature 2) KR 10-1409390 B1
(Patent literature 3) KR 10-0993623 B1

### Non-Patent Literatures

(Non-Patent Literature 1) Dobrynin, Danil, et al. “Physical and biological mechanisms of direct plasma interaction with living tissue.” New Journal of Physics 11.11 (2009): 115020.
(Non-Patent Literature 2) Ahn, Hak Jun, et al. “Targeting cancer cells with reactive oxygen and nitrogen species generated by atmospheric-pressure air plasma.” PloS one 9.1 (2014): e86173.
(Non-Patent Literature 3) Kang, S. U., et al. “Nonthermal plasma induces head and neck cancer cell death: the potential involvement of mitogen-activated protein kinase-dependent mitochondrial reactive oxygen species.” Cell Death and Disease 5 (2014): e1056.
(Non-Patent Literature 4) Ahn, Hak Jun, et al. “Atmospheric-pressure plasma jet induces apoptosis involving mitochondria via generation of free radicals.” PloS one 6.11 (2011): e28154.
(Non-Patent Literature 5) Kim, Kangil, et al. “Cellular membrane collapse by atmospheric-pressure plasma jet.” Applied Physics Letters 104.1 (2014): 013701.
(Non-Patent Literature 6) Lee, Changmin, et al. “Stability improvement of nonthermal atmospheric-pressure plasma jet using electric field dispersion.” Microelectronic Engineering 145 (2015): 153-159.

### DISCLOSURE

### Technical Problem

Therefore, the inventors confirmed a cancer cell killing effect and a dermal cell regeneration effect of plasma-activated liquid prepared by dissolving plasma generated using an atmospheric-pressure and low-temperature microplasma jet in a solution, and these effects can be maintained even when the plasma-activated liquid is stored in a freezer or a cold chamber, or at room temperature for a long time, and thus the present invention was completed.

Accordingly, an aspect of the present invention is directed to providing plasma-activated liquid prepared in the form capable of maintaining a therapeutic effect even when stored for a long time.

An aspect of the present invention is also directed to providing a skin regeneration or whitening composition, which includes the plasma-activated liquid as an active ingredient.

An aspect of the present invention is also directed to providing a wound healing composition, which includes the plasma-activated liquid as an active ingredient.

Another aspect of the present invention is also directed to providing a composition for preventing and treating cancer, which includes the plasma-activated liquid as an active ingredient.

### Technical Solution

In one aspect, the present invention provides a cosmetic composition for skin regeneration or whitening, which includes plasma-activated liquid as an active ingredient.

In another aspect, the present invention provides a pharmaceutical composition for skin regeneration or wound healing, which includes plasma-activated liquid as an active ingredient.

In still another aspect, the present invention provides a skin regeneration method, which includes administering an effective amount of plasma-activated liquid to a subject in need of skin regeneration.

In yet another aspect, the present invention provides a skin whitening method, which includes administering an effective amount of plasma-activated liquid to a subject in need of skin whitening.

In yet another aspect, the present invention provides a wound healing method, which includes administering an effective amount of plasma-activated liquid to a subject in need of wound healing.

In yet another aspect, the present invention provides a use of plasma-activated liquid as an active ingredient for a cosmetic composition for skin regeneration or whitening.

In yet another aspect, the present invention provides a use of plasma-activated liquid as an active ingredient for a pharmaceutical composition for skin regeneration or wound healing.

In yet another aspect, the present invention provides a pharmaceutical composition for preventing and treating cancer, which includes plasma-activated liquid as an active ingredient.

In yet another aspect, the present invention provides a health functional food for preventing and alleviating cancer, which includes plasma-activated liquid as an active ingredient.

In yet another aspect, the present invention provides a method of treating cancer, which includes administering an effective amount of plasma-activated liquid to a subject in need of cancer treatment.

In yet another aspect, the present invention provides a method of preventing cancer, which includes administering an effective amount of plasma-activated liquid to a subject in need of cancer prevention.

In yet another aspect, the present invention provides a use of plasma-activated liquid as an active ingredient for a pharmaceutical composition for preventing and treating cancer.

In yet another aspect, the present invention provides a use of plasma-activated liquid as an active ingredient for a health functional food for preventing and alleviating cancer.

In an exemplary embodiment of the present invention, the plasma-activated liquid may be prepared by jetting plasma generated in an atmospheric-pressure plasma jet onto an ion-containing solution, and the ion-containing solution may be one or more of water, a skin tonic, a culture medium and a buffer solution.

In an exemplary embodiment of the present invention, the plasma-activated liquid can be stored in a freezer at −20 to 0° C.; in a cold chamber at 0.1 to 10° C.; or at room temperature of 10 to 40° C., and the storage may be maintained for a day to 6 months.

In an exemplary embodiment of the present invention, the cancer may be any one or more selected from the group consisting of cervical cancer, lung cancer, breast cancer, colon cancer and skin cancer.

### Advantageous Effects

Accordingly, the present invention provides a skin regeneration or whitening composition; a wound healing composition; and a composition for preventing and treating cancer, each of which includes plasma-activated liquid having a form capable of being stored for a long period of time as an active ingredient.

In the present invention, since plasma-activated liquid prepared by dissolving plasma generated using an atmospheric-pressure and low-temperature microplasma jet in a solution not only exhibits a cancer cell killing effect at a level similar to gas-phase plasma in the conventional art, but also maintains the cancer cell killing effect even when the plasma-activated liquid is stored in a freezer or a cold chamber for 6 months, the plasma-activated liquid is suitable for long-term storage and thus can be effectively used in the biopharmaceutical field. In addition, as it was confirmed that the plasma-activated liquid of the present invention exhibits a regeneration effect on wound-induced dermal cells, and also exhibits a significant skin regeneration effect even when stored in a cold chamber or at room temperature for 6 months, the plasma-activated liquid of the present invention is suitable for long-term storage and thus can be used as an active ingredient for a composition for cancer treatment or a cosmetic composition for skin regeneration.

### DESCRIPTION OF DRAWINGS

FIGS. 1A-1C show information associated with an atmospheric-pressure and low-temperature microplasma jet device for producing plasma-activated liquid.

FIG. 1A shows an electrode substrate manufactured to be mounted in the atmospheric-pressure and low-temperature microplasma jet.

FIG. 1B shows an optical emission spectrum of gas-phase plasma formed by the atmospheric-pressure and low-temperature microplasma jet.

FIG. 1C shows a change in concentration of nitrogen gas emitted from the atmospheric-pressure and low-temperature microplasma jet.

FIGS. 2A-2B show an anticancer activity of direct plasma using conventional gas-phase plasma and plasma-activated liquid of the present invention:

FIG. 2A is a set of diagrams showing cell killing effects on HeLa cells treated with the direct plasma and the plasma-activated liquid, respectively, observed by fluorescence microscopy; and

FIG. 2B is the graph obtained by quantifying the cell killing effects.

FIGS. 3A-3D show a long-term storage suitability of plasma-activated liquid (liquid plasma; LP) of the present invention:

FIGS. 3A and 3B are diagrams showing a cancer cell killing effect of plasma-activated liquid after storage in a freezer for 24 or 48 hours; and

FIGS. 3C and 3D are diagrams showing cancer cell death types caused by plasma-activated liquid after storage in a freezer for 1 or 6 months.

FIGS. 4A-4B show ROS and RNS generated in plasma-activated liquid stored in a freezer for an extended period of time:

FIG. 4A shows a concentration of ROS generated in plasma-activated liquid after storage in a freezer for 1 or 6 months; and

FIG. 4B shows a concentration of RNS generated in plasma-activated liquid after storage in a freezer for 1 or 6 months.

FIG. 5 shows a regeneration effect of plasma-activated liquid of the present invention on human epidermal keratinocyte (HEK) cells.

FIG. 6 is a set of diagrams showing a regeneration effect of plasma-activated liquid stored in a cold chamber or at room temperature for 6 months on HEK cells.

FIG. 7 is a graph showing a dermal cell regeneration effect after plasma-activated liquid generated using a skin tonic or PBS is stored for a long time.

FIG. 8 is a graph showing skin whitening and skin regeneration effects of plasma-activated liquid stored for a long time of 6 months.

### MODES OF THE INVENTION

Hereinafter, the present invention will be described in detail.

As described above, although room-temperature, atmospheric-pressure plasma can be effectively used in various biopharmaceutical fields such as cancer treatment, gas-phase plasma is not effective for administration and delivery into the body, and is not suitable for long-term storage, and thus its application is limited.

In the present invention, since plasma-activated liquid prepared by dissolving a plasma generated using an atmospheric-pressure and low-temperature microplasma jet in a solution may not only exhibit a cancer cell killing effect at a level similar to gas-phase plasma of the conventional art, but also maintain the cancer cell killing effect even when stored in a freezer or a cold chamber for 6 months, the plasma-activated liquid is suitable for long-term storage and thus can be effectively used in the biopharmaceutical field. In addition, as it is confirmed that the plasma-activated liquid of the present invention exhibits a regeneration effect on wound-induced dermal cells, and also exhibits a significant skin regeneration effect even when stored in a cold chamber or at a room temperature for 6 months, the plasma-activated liquid of the present invention is suitable for long-term storage and can be used as an active ingredient for a composition for treating cancer or a cosmetic composition for skin regeneration.

Therefore, the present invention provides a cosmetic composition for skin regeneration or whitening, which includes plasma-activated liquid as an active ingredient.

The “plasma-activated liquid (liquid plasma)” used herein may be prepared by jetting plasma generated in an atmospheric-pressure plasma jet onto an ion-containing solution. Specifically, the plasma-activated liquid is preferably prepared by generating plasma using an atmospheric-pressure plasma jet through microelectromechanical system (MEMS) technology; and applying the plasma to a solution for treatment, and more specifically, the plasma-activated liquid is prepared using a method disclosed in Korean Patent No. 10-1409390.

The atmospheric-pressure plasma jet consists of an electrode used as an anode, a gas injection tube used as a cathode, a porous insulating material, a protection tube and an insulating case. In the step of preparing plasma-activated liquid according to the present invention, nickel (Ni) is preferable for the electrode used as an anode, a stainless steel cathode is preferably used as the gas injection tube used as a cathode, and more specifically, the atmospheric-pressure plasma jet is most preferably the device disclosed in Korean Patent No. 10-1001477, but the present invention is not limited thereto.

In a plasma discharge step through the atmospheric-pressure plasma jet for preparing the “plasma-activated liquid (liquid plasma)” of the present invention, a gas injected into the device is preferably nitrogen (N₂), and the gas is preferably injected at a flow rate of 5 to 15 L/min. When the nitrogen gas is injected, ionic particles and radicals may be included so that ROS and RNS are easily generated in the plasma-activated liquid. A voltage applied to the atmospheric-pressure plasma jet is preferably 5 to 20 kVp-p, but the present invention is not limited thereto, and a voltage in a range, recognized in the art, in which the plasma-activated liquid can be effectively prepared, may be applied.

The “plasma-activated liquid (liquid plasma)” of the present invention may be prepared by jetting plasma onto an ion-containing solution. The solution may be, but is not particularly limited to, water, a skin tonic, a culture medium or a buffer solution, which contains ions, and any liquid agent which easily and stably includes plasma ion particles and radicals can be used without limitation.

The “plasma-activated liquid (liquid plasma)” of the present invention is suitable for long-term storage, compared with conventional gas-phase plasma. Specifically, the plasma-activated liquid of the present invention may be stored in a freezer, in a cold chamber or at room temperature for 1 day to 6 months. The freezer storage is preferably performed at −20 to 0° C.; cold storage is performed at 0.1 to 10° C.; and room-temperature storage is performed at 10 to 40° C., but the present invention is not limited thereto. Compared with fresh plasma-activated liquid, which is not stored, a storage period and a temperature may be adjusted within a range maintaining similar levels of anticancer activity and skin regeneration effect.

The cosmetic composition may be used for an external use for the skin or orally administered, but the present invention is not particularly limited thereto.

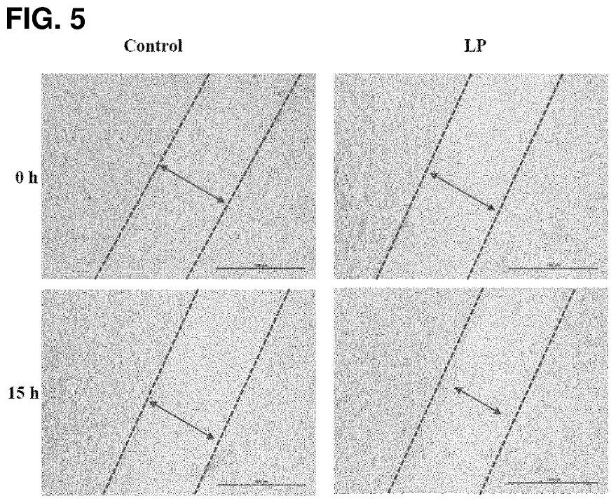

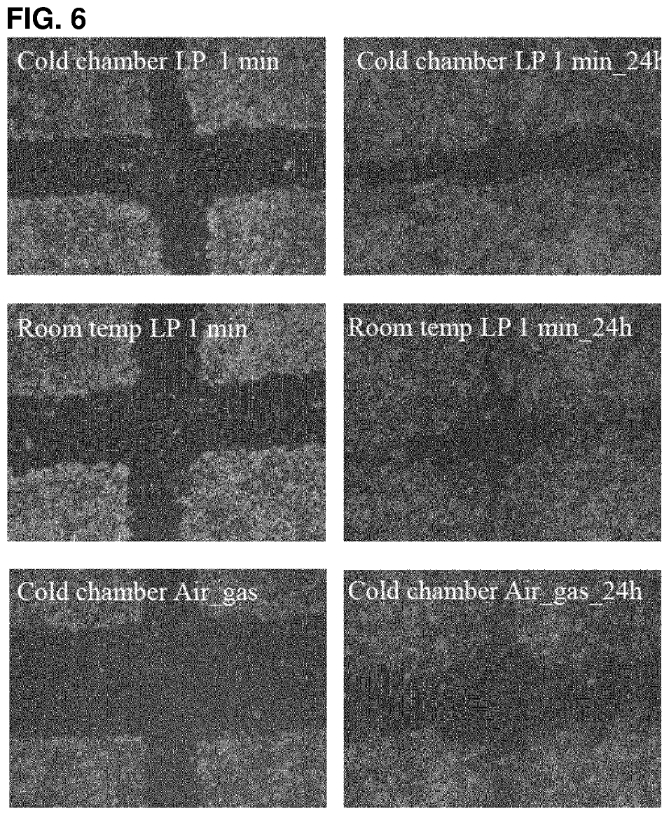

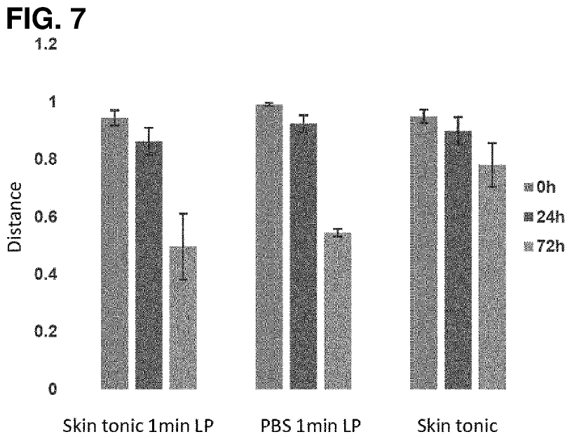

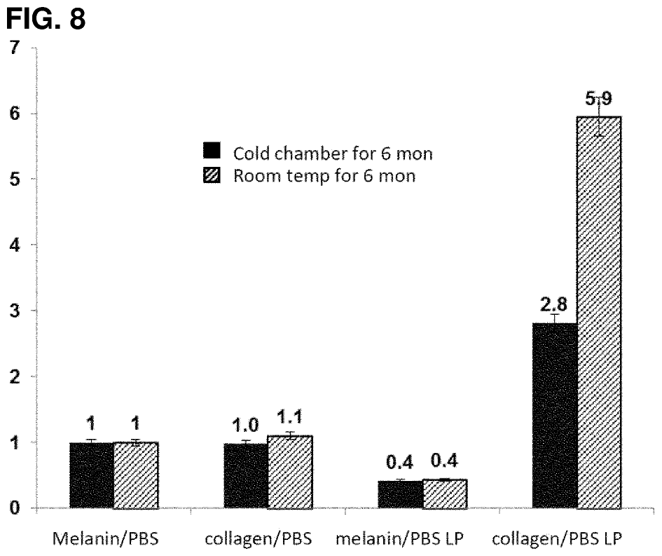

In an exemplary embodiment of the present invention, as a result of confirming a dermal cell regeneration effect using plasma-activated liquid, the inventors confirmed that plasma-activated liquid preparing using DEME, a skin tonic or PBS can exhibit a significant wound healing effect on HEK cells (FIG. 5), and the dermal cell regeneration effect is also maintained at a similar level even using plasma-activated liquid stored in a cold chamber or at room temperature for 6 months (FIGS. 6 and 7). In addition, it was confirmed that the plasma-activated liquid of the present invention exhibits an effect of increasing collagen synthesis and inhibiting melanin synthesis in a malignant skin cancer cell line (FIG. 8).

Therefore, as it was confirmed that the plasma-activated liquid of the present invention exhibits a regeneration effect on wound-induced dermal cells, and plasma-activated liquid stored in a cold chamber or at room temperature for 6 months can also exhibit a significant skin regeneration effect, the plasma-activated liquid of the present invention is suitable for long-term storage and thus can be used as an active ingredient for a composition for treating cancer or a composition for skin regeneration.

While the cosmetic composition of the present invention may contain plasma-activated liquid as an active ingredient, a cosmetic composition for skin care products (a skin tonic, a cream, an essence, a face cleanser such as a cleansing foam and a cleansing water, a pack, and a body oil) may be prepared by generating plasma itself as a composition in the form of plasma-activated liquid. In addition, the cosmetic composition of the present invention may be prepared in the form of a cosmetic composition for skin care products, a cosmetic composition for make-up products (a foundation, a lipstick, a mascara, and a make-up base), a composition for hair products (a shampoo, a rinse, a hair conditioner, and a hair gel) or a soap, in addition to dermatologically acceptable excipients.

The excipients may include, but are not limited to, for example, an emollient, a skin penetration enhancer, a pigment, a flavoring agent, an emulsifier, a thickening agent, and a solvent. The excipients may further include a fragrance, a colorant, an antibacterial agent, an antioxidant, a preservative and a moisturizing agent, and for improvement of physical properties, include a thickener, inorganic salts, and a synthetic high-molecular substance. For example, when a face cleanser and a soap are manufactured using the cosmetic composition of the present invention, they can be easily manufactured by adding the plasma-activated liquid to a conventional face cleanser and soap base. When a cream is manufactured, the cream may be manufactured by adding plasma-activated liquid or a salt thereof to a general oil-in-water (O/W) cream base. Here, synthetic or natural materials such as proteins, minerals, vitamins, etc. may be further added to improve physical properties, in addition to a fragrance, a chelating agent, a colorant, an antioxidant, and a preservative.

A content of the plasma-activated liquid contained in the cosmetic composition of the present invention is preferably 0.001 to 10 wt %, and more preferably, 0.01 to 5 wt % with respect to the total weight of the composition, but the present invention is not limited thereto. When the content is less than 0.001 wt %, a desired antiaging or antiwrinkle effect cannot be expected, and when the content is more than 10 wt %, it can be difficult to prepare plasma-activated liquid due to safety or formulation problems.

In addition, the present invention provides a pharmaceutical composition for skin regeneration or wound healing, which includes plasma-activated liquid as an active ingredient.

The “plasma-activated liquid (liquid plasma)” of the present invention may be prepared by jetting plasma generated in an atmospheric-pressure plasma jet onto an ion-containing solution. Specifically, the plasma-activated liquid is preferably prepared by generating plasma using an atmospheric-pressure plasma jet through MEMS technology; and applying the plasma to a solution for treatment, and more specifically, the plasma-activated liquid is prepared using a method disclosed in Korean Patent No. 10-1409390.

The atmospheric-pressure plasma jet consists of an electrode used as an anode, a gas injection tube used as a cathode, a porous insulating material, a protection tube and an insulating case. In the step of preparing plasma-activated liquid according to the present invention, nickel (Ni) is preferable for the electrode used as an anode, a stainless steel cathode is preferably used as the gas injection tube used as a cathode, and more specifically, the atmospheric-pressure plasma jet is most preferably the device disclosed in Korean Patent No. 10-1001477, but the present invention is not limited thereto.

In a plasma discharge step through the atmospheric-pressure plasma jet for preparing the “plasma-activated liquid (liquid plasma)” of the present invention, a gas injected into the device is preferably nitrogen (N₂), and the gas is preferably injected at a flow rate of 5 to 15 L/min. When the nitrogen gas is injected, ionic particles and radicals may be included so that ROS and RNS are easily generated in the plasma-activated liquid. A voltage applied to the atmospheric-pressure plasma jet is preferably 5 to 20 kVp-p, but the present invention is not limited thereto, and a voltage in a range, recognized in the art, in which the plasma-activated liquid can be effectively prepared, may be applied.

The “plasma-activated liquid (liquid plasma)” of the present invention may be prepared by jetting plasma onto an ion-containing solution. The solution may be, but is not particularly limited to, water, a skin tonic, a culture medium or a buffer solution, which contains ions, and any liquid agent which easily and stably includes plasma ion particles and radicals can be used without limitation.

The “plasma-activated liquid (liquid plasma)” of the present invention is suitable for long-term storage, compared with conventional gas-phase plasma. Specifically, the plasma-activated liquid of the present invention may be stored in a freezer, in a cold chamber or at room temperature for 1 day to 6 months. The freezer storage is preferably performed at −20 to 0° C.; cold storage is performed at 0.1 to 10° C.; and room-temperature storage is performed at 10 to 40° C., but the present invention is not limited thereto. Compared with fresh plasma-activated liquid, which is not stored, a storage period and a temperature may be adjusted within a range maintaining similar levels of anticancer activity and skin regeneration effect.

The pharmaceutical composition for skin regeneration or wound healing according to the present invention may contain plasma-activated liquid only, or one or more types of active ingredients exhibiting a function similar to the plasma-activated liquid. The additional components may impart further improvement in the skin regeneration effect of the composition according to the present invention. When the additional components are used, dermal safety according to combinational use, ease of formulation, and the stability of active ingredients can be considered.

In addition, the pharmaceutical composition for skin regeneration or wound healing according to the present invention may further include pharmaceutically acceptable carriers.

The pharmaceutically acceptable carriers may further include, for example, carriers for oral administration or carriers for non-oral administration. The carriers for oral administration may include lactose, starch, cellulose derivatives, magnesium stearate, and stearic acid. In addition, the carriers for non-oral administration may include water, suitable oil, saline, aqueous glucose, and glycol. In addition, the pharmaceutically acceptable carriers may further include a stabilizer and a preservative. The suitable stabilizer may be an antioxidant such as sodium hydrogen sulfite, sodium sulfite, or ascorbic acid. As a suitable preservative, there is benzalkonium chloride, methyl- or propyl-paraben, or chlorobutanol. For other pharmaceutically acceptable carriers, the following literature (Remington's Pharmaceutical Sciences, 19th ed., Mack Publishing Company, Easton, Pa., 1995) may be referenced.

The pharmaceutical composition of the present invention may be administered to mammals including a human by any method. For example, the pharmaceutical composition of the present invention may be orally or non-orally administered, and the non-oral administration method may be, but is not limited to, intravenous, intramuscular, intraarterial, intramedullary, intrathecal, intracardiac, transdermal, subcutaneous, intraperitoneal, intranasal, intestinal, local, sublingual or rectal administration.

The pharmaceutical composition of the present invention may be formulated as an agent for oral or non-oral administration according to an administration route as described above. In formulation, one or more selected from the group consisting of a buffer (e.g., saline or PBS), an antioxidant, a bacteriostat, a chelating agent (e.g., EDTA or glutathione), a filler, an expander, a binder, an adjuvant (e.g., aluminum hydroxide), a suspending agent, a thickening agent, a disintegrating agent or a surfactant, a diluent or an excipient may be used.

A solid agent for oral administration may include a tablet, a pill, a powder, a granule, a liquid, a gel, a syrup, a slurry, a suspension or a capsule, and such solid agent may be prepared by mixing at least one or more excipients, for example, starch (corn starch, wheat starch, rice starch, potato starch, etc.), calcium carbonate, sucrose, lactose, dextrose, sorbitol, mannitol, xylitol, erythritol, maltitol, cellulose, methyl cellulose, sodium carboxy methylcellulose and hydroxypropylmethyl-cellulose or gelatin with the pharmaceutical composition of the present invention. For example, the active ingredient may be mixed with a solid excipient, grinded, mixed with a suitable additive, and processed into a powder mixture, and thereby a tablet or sugar-coated tablet may be obtained.

In addition to simple excipients, a lubricant such as magnesium stearate talc is used. Liquid agents for oral administration include a suspending agent, a liquid for internal use, an emulsion, and a syrup, and in addition to a simple diluent frequently used, such as water or liquid paraffin, various excipients, for example, a wetting agent, a sweetening agent, a fragrance or a flavoring agent and a preservative may be included.

In addition, in some cases, crosslinked polyvinylpyrrolidone, agar, alginic acid or sodium alginate may be added as a disintegrant, and an anti-coagulant, a lubricant, a wetting agent, a fragrance, an emulsifier and a preservative may be additionally included.

For non-oral administration, the pharmaceutical composition of the present invention may be formulated in the form of an injection, a transdermal administration agent and a nasal inhalant according to a method known in the art. The injection must be sterilized and should be protected from contamination of microorganisms such as bacteria and fungi. Examples of suitable carriers for injections may be, but are not limited to, for example, water, ethanol, a polyol (e.g., glycerol, propylene glycol, liquid polyethylene glycol, etc.), a mixture thereof, and/or a solvent or dispersion medium including vegetable oil. More preferably, as the suitable carrier, a Hank's solution, a Ringer's solution, triethanolamine-containing phosphate buffered saline (PBS) or injectable sterile water, or an isotonic solution such as 10% ethanol, 40% propylene glycol or 5% dextrose may be used. To protect the injection from microbial contamination, various antibacterial agents and antifungal agents such as paraben, chlorobutanol, phenol, sorbic acid, thimerosal, etc. may be further included. In addition, in most cases, the injection may further include an isotonic agent such as sugar or sodium chloride.

The transdermal administration agent is an ointment, a cream, a lotion, a gel, a liquid for external use, a paste, a liniment, or an aerogel. Here, the “transdermal administration” refers to intradermal delivery of an effective amount of an active ingredient contained in a pharmaceutical composition by administering the pharmaceutical composition locally onto the skin.

In the case of the inhalant, the compound used according to the present invention may be conveniently delivered in the form of a pressurized pack, spray gun or an aerosol spray using a suitable propellant, for example, dichlorofluoromethane, trichlorofluoromethane, dichlorotetrafluoroethane, carbon dioxide or a different suitable gas. In the case of pressurized aerosol, a dosage unit may be determined by providing a valve through which a measured amount of the pressurized aerosol is delivered. For example, a gelatin capsule and a cartridge, which are used in an inhaler or insufflator may be formulated to contain a powder mixture of a compound, and a suitable powder base such as lactose or starch. The formulations for non-oral administration are disclosed in protocols generally known in all pharmaceutical chemistry fields (Remington's Pharmaceutical Science, 15th Edition, 1975. Mack Publishing Company, Easton, Pa. 18042, Chapter 87: Blaug, Seymour).

The pharmaceutical composition for skin regeneration or wound healing according to the present invention may provide a preferable skin regeneration effect when an effective amount of plasma-activated liquid is included. The term “effective amount” used herein refers to an amount capable of exhibiting a response at a higher level than a negative control, and preferably, an amount sufficient for enhancing muscle function. The pharmaceutical composition of the present invention may include 0.01 to 99.99% of plasma-activated liquid, and the remaining amount may consist of pharmaceutically acceptable carriers. The effective amount of the plasma-activated liquid included in the pharmaceutical composition of the present invention may vary according to a product type formed of the composition.

The total effective amount of the pharmaceutical composition of the present invention may be administered to a patient at a single dose, and may be administered by a fractionated treatment protocol used for long-term administration in multiple doses. It is important to administer an amount that achieves the greatest effect with the least amount without side effects by considering all of the above-mentioned factors, and the amount may be easily determined by those of ordinary skill in the art.

The pharmaceutical composition of the present invention may contain a different content of an active ingredient depending on the severity of a disease. In the non-oral administration, the pharmaceutical composition of the present invention is preferably administered one to several times daily at 0.01 to 50 mg, and more preferably, 0.1 to 30 mg per kg of a body weight with respect to the plasma-activated liquid, and in oral administration, the pharmaceutical composition of the present invention is preferably administered one to several times daily at 0.01 to 100 mg, and more preferably, 0.01 to 10 mg per kg of a body weight with respect to the plasma-activated liquid. However, an effective dose of the plasma-activated liquid is determined by considering various parameters such as a patient's age, body weight, health condition, and sex, the severity of a disease, diet and an excretion rate, as well as the administration route of the pharmaceutical composition and the number of treatment cycles, and thus, considering these parameters, a suitable effective dose of the plasma-activated liquid according to a specific use for preventing and treating cancer can be determined by those of ordinary skill in the art. As long as the pharmaceutical composition according to the present invention exhibits the effect of the present invention, its formulation, administration route and administration method are not particularly limited.

The pharmaceutical composition for skin regeneration or wound healing according to the present invention may be used alone or in combination with methods using surgery, radiation therapy, hormone therapy, chemotherapy or biological reaction adjustment.

The pharmaceutical composition for skin regeneration or wound healing according to the present invention may also be provided in a formulation of a formulation for external use, which includes plasma-activated liquid as an active ingredient.

When the pharmaceutical composition for skin regeneration or wound healing according to the present invention is used as a dermal formulation for external use, additives conventionally used in the field of dermatology, which are the same as a random component conventionally used in a dermal formulation for external use, for example, an adipose substance, an organic solvent, a solubilizer, a concentrating agent and a gelling agent, a softening agent, an antioxidant, a suspending agent, a stabilizer, a foaming agent, a flavoring agent, a surfactant, water, an ionic emulsifier, a non-ionic emulsifier, a filler, a sequestering agent, a chelating agent, a preservative, a vitamin, a blocking agent, a wetting agent, an essential oil, a dye, a pigment, a hydrophilic active agent, a lipophilic active agent or lipid vesicles, may be contained. In addition, the components may be introduced at an amount generally used in dermatology field.

When the pharmaceutical composition for skin regeneration or wound healing according to the present invention is provided as a dermal formulation for external use, the composition may have a formulation such as an ointment, a patch, a gel, a cream or a spray, but the present invention is not limited thereto.

In addition, the present invention provides a pharmaceutical composition for preventing and treating cancer, which includes plasma as an active ingredient.

In addition, the present invention provides a health functional food for preventing and alleviating cancer, which includes plasma-activated liquid as an active ingredient.

The “plasma-activated liquid (liquid plasma)” of the present invention may be prepared by jetting plasma generated in an atmospheric-pressure plasma jet onto an ion-containing solution. Specifically, the plasma-activated liquid is preferably prepared by generating plasma using an atmospheric-pressure plasma jet through microelectromechanical system (MEMS) technology; and applying the plasma to a solution for treatment, and more specifically, the plasma-activated liquid is prepared using a method disclosed in Korean Patent No. 10-1409390.

The atmospheric-pressure plasma jet consists of an electrode used as an anode (anode), a gas injection tube used as a cathode (cathode), a porous insulating material, a protection tube and an insulating case. In the step of preparing plasma-activated liquid according to the present invention, nickel (Ni) is preferable for the electrode used as an anode, a stainless steel cathode is preferably used as the gas injection tube used as a cathode, and more specifically, the atmospheric-pressure plasma jet is most preferably the device disclosed in Korean Patent No. 10-1001477, but the present invention is not limited thereto.

In a plasma discharge step through the atmospheric-pressure plasma jet for preparing the “plasma-activated liquid (liquid plasma)” of the present invention, a gas injected into the device is preferably nitrogen (N₂), and the gas is preferably injected at a flow rate of 5 to 15 L/min. When the nitrogen gas is injected, ionic particles and radicals may be included so that ROS and RNS are easily generated in the plasma-activated liquid. A voltage applied to the atmospheric-pressure plasma jet is preferably 5 to 20 kVp-p, but the present invention is not limited thereto, and a voltage in a range, recognized in the art, in which the plasma-activated liquid can be effectively prepared, may be applied.

The “plasma-activated liquid (liquid plasma)” of the present invention may be prepared by jetting plasma onto an ion-containing solution. The solution may be, but is not particularly limited to, water, a skin tonic, a culture medium or a buffer solution, which contains ions, and any liquid agent which easily and stably includes plasma ion particles and radicals can be used without limitation.

The “plasma-activated liquid (liquid plasma)” of the present invention is suitable for long-term storage, compared with conventional gas-phase plasma. Specifically, the plasma-activated liquid of the present invention may be stored in a freezer, in a cold chamber or at room temperature for 1 day to 6 months. The freezer storage is preferably performed at −20 to 0° C.; cold storage is performed at 0.1 to 10° C.; and room-temperature storage is performed at 10 to 40° C., but the present invention is not limited thereto. Compared with fresh plasma-activated liquid, which is not stored, a storage period and a temperature may be adjusted within a range maintaining similar levels of anticancer activity and skin regeneration effect.

The “cancer” used herein is preferably any one or more selected from the group consisting of cervical cancer, lung cancer, breast cancer, colon cancer and skin cancer, and more preferably, any one or two of cervical cancer and skin cancer, but the present invention is not limited thereto. Any cancer known in the art that the gas plasma according to the conventional art can exhibit anticancer activity can be applied to the present invention.

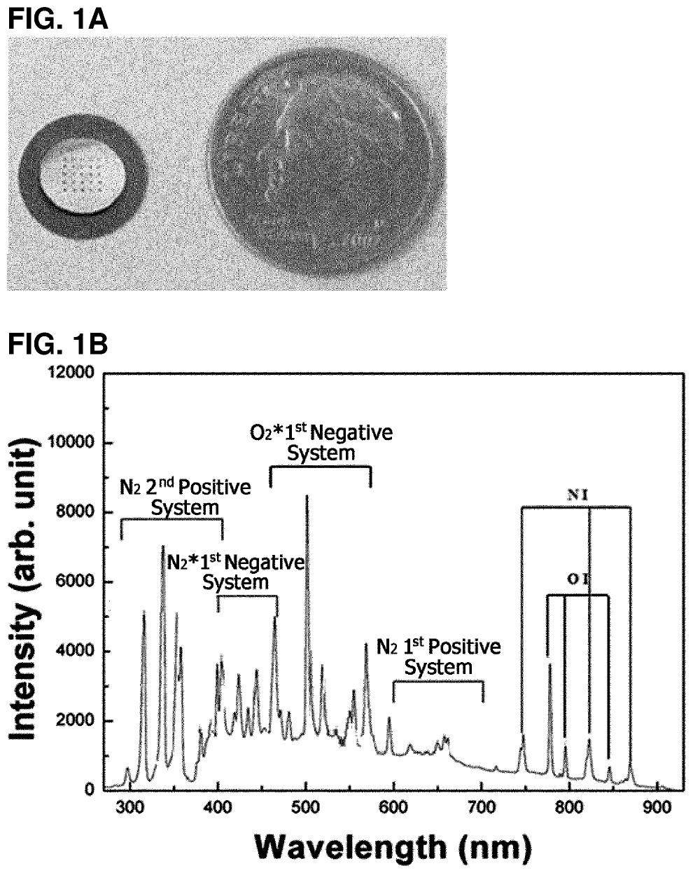

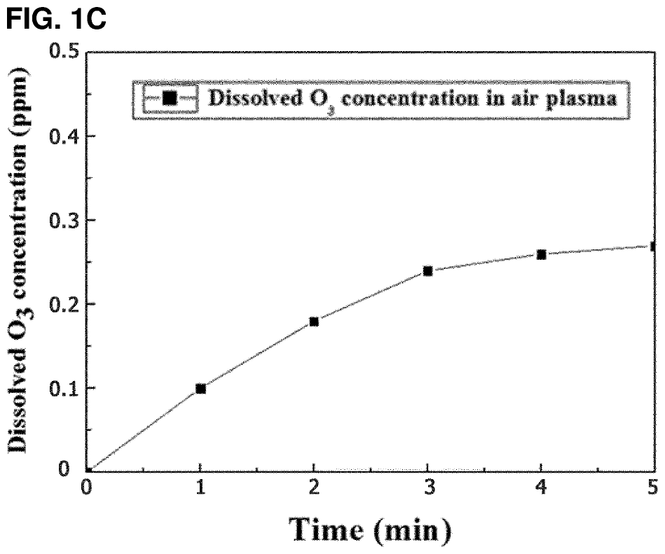

In an exemplary embodiment of the present invention, the inventors produced plasma-activated liquid using DMEM, a skin tonic and PBS to overcome the limitations of storage and in-vivo delivery of conventional gas-phase atmospheric-pressure low-temperature plasma (FIGS. 1A-1C).

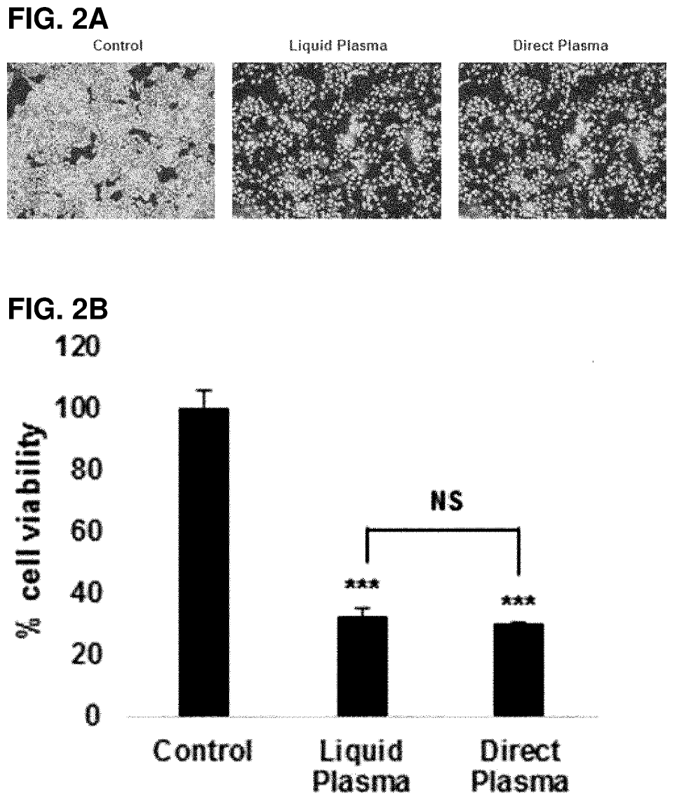

In addition, the inventors confirmed that the plasma-activated liquid produced using the DMEM can kill HeLa cells at a level similar to the cancer cell killing effect exhibited by conventional gas-phase plasma (FIGS. 2A and 2B).

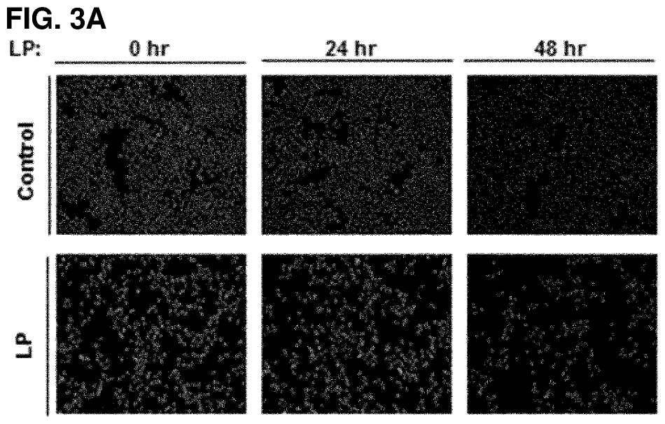

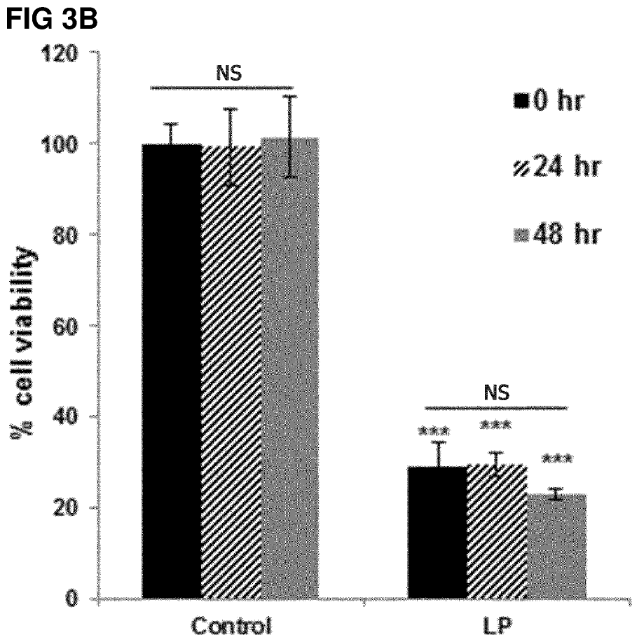

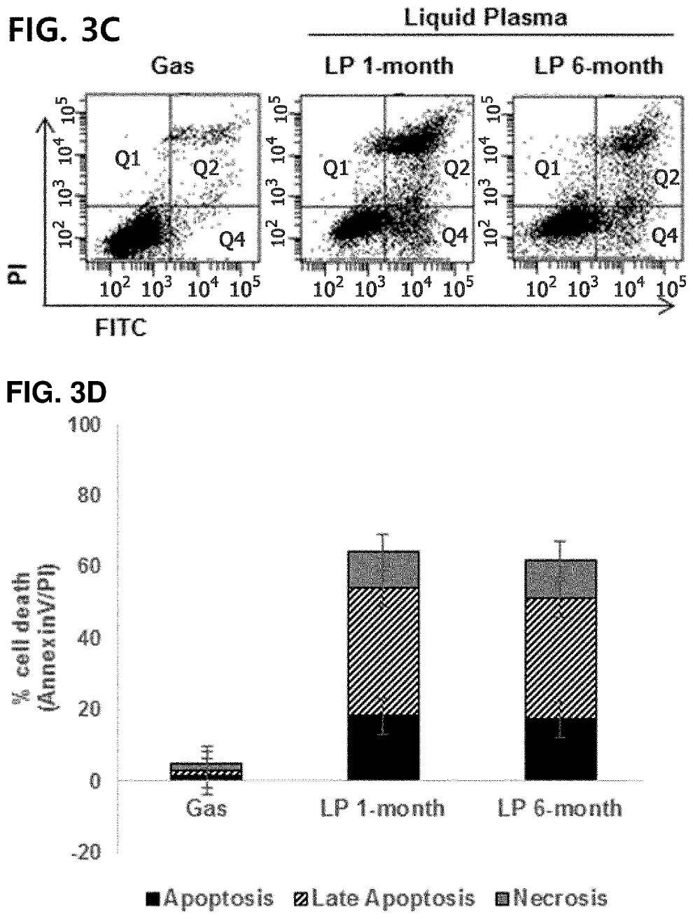

In addition, as a result of examining a cancer cell killing effect by freezing plasma-activated liquid at −20° C. for 1- to 6-month storage and thawing the frozen plasma-activated liquid to confirm whether the plasma-activated liquid of the present invention is suitable for long-term storage, the inventors confirmed that the cancer cell killing effect is exhibited at a level similar to that shown in fresh plasma-activated liquid immediately used after preparation (FIGS. 3A and 3B), and such effect is caused by apoptosis (FIGS. 3C and 3D).

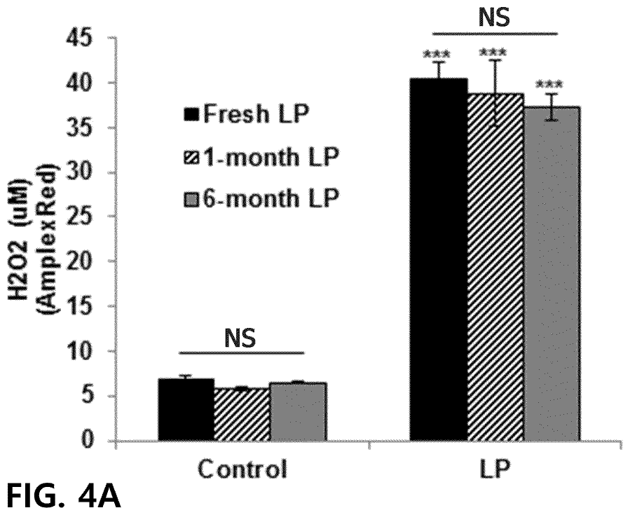

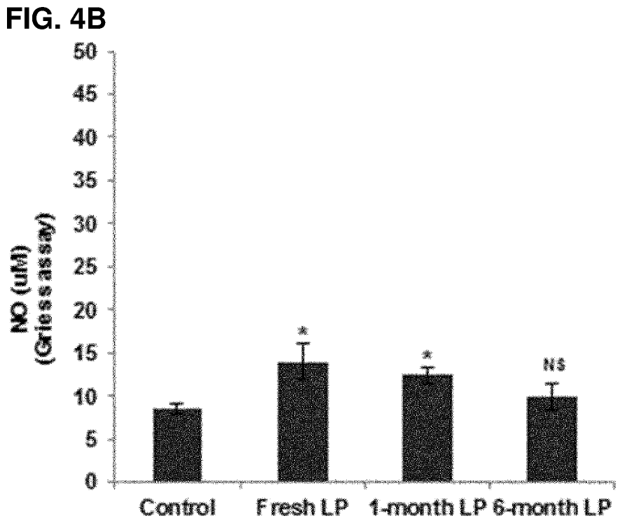

In addition, as a result of confirming whether the apoptosis of cancer cells is caused by plasma-activated liquid, the inventors confirmed that, in both fresh plasma-activated liquid and long-term stored plasma-activated liquid, ROS and RNS are produced at similar levels, thereby inducing apoptosis by the plasma-activated liquid (FIGS. 4A and 4B).

Accordingly, in the present invention, plasma-activated liquid prepared by dissolving the plasma generated using an atmospheric-pressure and low-temperature microplasma jet in a solution not only exhibits a cancer cell killing effect at a level similar to conventional gas-phase plasma, but also maintains the cancer cell killing effect after being stored in a freezer or a cold chamber for 6 months. Therefore, the plasma-activated liquid can be suitable for long-term storage and effectively used in the biopharmaceutical field relating to pharmaceutical compositions for preventing and treating cancer.

The food composition of the present invention includes all types of functional foods, nutritional supplements, health foods, food additives and feeds, and is used for a human or animals including livestock. The above-mentioned types of food compositions may be prepared in various forms according to a conventional method known in the art.

The food composition according to the present invention may be prepared in various forms according to a conventional method known in the art. The food composition of the present invention may be prepared as general food by adding the plasma-activated liquid of the present invention to any one of beverages (including alcohol beverages), fruit and processed food thereof (e.g., canned fruit, bottled food, jam, marmalade, etc.), fish, meat and processed food thereof (e.g., ham, sausage, corn beef, etc.), bread and noodles (e.g., udon, buckwheat noodles, ramen, spaghetti, macaroni, etc.), fruit juices, various drinks, cookies, sugar, dairy products (e.g., butter, cheese, etc.), edible vegetable oil, margarine, vegetable protein, retort food, frozen food, or various kinds of seasoning (e.g., bean paste, soy sauce, sauces, etc.), but the present invention is not limited thereto. In addition, the food composition of the present invention may be prepared as a nutritional supplement by adding the plasma-activated liquid of the present invention to capsules, tablets, pills or the like, but the present invention is not limited thereto. In addition, as a health functional food, the plasma-activated liquid itself may be ingested through liquidation, granulation, encapsulation or pulverization to drink it after being produced in the form of tea, juice or a drink (as a healthy drink), but the present invention is not limited thereto. In addition, to be used in the form of a food additive, the plasma-activated liquid of the present invention may be produced in the form of a powder or concentrate. In addition, to produce a composition, the plasma-activated liquid of the present invention may be mixed with an active ingredient known to have an anticancer effect.

When the plasma-activated liquid of the present invention is used in a healthy drink, the healthy drink composition may contain various favoring agents or natural carbohydrates as additional components, like a conventional drink. The above-mentioned natural carbohydrate may be a monosaccharide such as glucose or fructose; a disaccharide such as maltose or sucrose; a polysaccharide such as dextrin or cyclodextrin; or a sugar alcohol such as xylitol, sorbitol or erythritol. As a sweetening agent, a natural sweetening agent such as a thaumatin or stevia extract; or a synthetic sweetening agent such as saccharin or aspartame may be used. A ratio of the natural carbohydrate may be generally approximately 0.01 to 0.04 g, and preferably 0.02 to 0.03 g based on 100 mL of the composition of the present invention.

In addition, the plasma-activated liquid of the present invention may be contained as an active ingredient of the food composition for preventing and alleviating cancer, and an amount thereof is an amount effective to achieve an anticancer action, and preferably, 0.01 to 100 wt % with respect to the total weight of the composition, but the amount is not particularly limited thereto. The food composition of the present invention may be prepared by mixing the plasma-activated liquid with a different active ingredient known to have an anticancer effect.

Other than these, the health functional food of the present invention may contain various nutrients, vitamins, electrolytes, flavoring agents, colorants, pectic acid or a salt thereof, alginic acid or a salt thereof, organic acids, a protective colloidal thickening agent, a pH adjuster, a stabilizer, a preservative, glycerin, alcohols or a carbonating agent. In addition, the health functional food of the present invention may contain fruit flesh for producing a natural fruit juice, a fruit juice drink, or a vegetable drink. These components may be used independently or in combination with each other. A ratio of the additive is not significantly important, but generally selected in the range of 0.01 to 0.1 part by weight with respect to 100 parts by weight of the composition of the present invention.

Hereinafter, the present invention will be described in further detail with reference to examples. The examples are merely provided to more fully describe the present invention, and it will be obvious to those of ordinary skill in the art that the scope of the present invention is not limited to the following examples.

### EXAMPLES

### Example 1

Preparation of Plasma-Activated Liquid

<1-1> Production of Microplasma Jet

To prepare plasma-activated liquid (liquid plasma) of the present invention from a gas-phase plasma jet, an atmospheric-pressure and low-temperature microplasma jet was manufactured using micromachining fabrication, this was used to form plasma-activated liquid. The atmospheric-pressure and low-temperature microplasma jet was manufactured in accordance with a known method (Patent Literature 1 or Non-Patent Literature 6). The device consisted of i) a nickel anode from which plasma was jetted; ii) a stainless steel cathode into which a gas flowed; and iii) a module case of an insulating layer connecting the anode with the cathode.

Specifically, first, a nickel anode was formed. A seed layer was formed by electroplating a titanium (Ti)-copper (Cu) film on a glass wafer. An entire anode was formed by patterning the glass wafer on which the seed layer through photolithography, and performing electroplating with nickel (Ni)-cobalt (Co). An anode substrate having a thickness of 80 μm was formed by nickel plating at a current density of 50 mA/cm² for 80 hours. After the nickel-cobalt electroplating, the plated glass wafer was planarized by polishing, thereby completing a nickel anode. The nickel anode was designed to have a round wafer shape with a diameter of 10 mm and have 5×5 (total of 25) pores through which plasma was jetted from each wafer. To confirm accurate capacitance and electronic density, the nickel anode was designed to have a gap of 100 to 300 lm. As the stainless steel cathode, a tube with a diameter of 1.5 mm was used, and each electrode was mounted in a plastic module case of an insulating layer, thereby completing a microplasma jet. The total size of the device was suitably adjusted according to an experimental environment.

As a result, a microplasma jet including an electrode substrate having a shape as shown in FIG. 1A was manufactured.

<1-2> Confirmation of Gas-Phase Plasma Generated in Microplasma Jet

Prior to the preparation of plasma-activated liquid, characteristics of gas-phase plasma generated in the microplasma jet manufactured in the present invention were identified.

Specifically, the microplasma jet manufactured in Example 1-1 was equipped with a power supply for a discharge experiment, and a voltage of 20 kVp-p and a frequency of 20 kHz were applied. In addition, nitrogen gas (N₂) was injected through the stainless steel cathode tube at a rate of 10 L/min, thereby producing gas-phase plasma. While the gas-phase plasma was produced, optical emission spectra were measured to identify ion particles and radicals of the produced plasma using optical emission spectroscopy (OES; SV 2100, K-MAC, Korea). The optical emission spectroscopy was performed to measure a spectrum by setting a wavelength range of 280 to 920 nm. In addition, the gas-phase plasma was jetted using a gas detector (MultiRAE, Rate Systems, USA), and then a change in concentration of nitrogen gas over time was examined.

As a result, as shown in FIGS. 1B and 1C, it was confirmed that a large amount of excited oxygen ions (O₂⁺) and excited nitrogen molecules of a donor, which serve to generate ROS and RNS in the gas-phase plasma produced by the microplasma jet of the present invention can be generated (FIG. 1B). In addition, when a nitrogen gas concentration was checked by a gas detector, NO gas was removed from the jetted gas-phase plasma for the first one minute from the initiation of the generation of a plasma, and then the gas concentration gradually recovered and was emitted at 35 to 40 ppm (FIG. 1C). To this end, it was confirmed that various nitrogen and oxygen species can be produced from the microplasma jet of the present invention.

<1-3> Preparation of Plasma-Activated Liquid Using Microplasma Jet

According to the conventional art, it has been reported that, since the gas-phase plasma produced using the jet has a room temperature, when directly jetted onto cells, the gas-phase plasma can induce cell death caused by apoptosis, and thus can be used for treatment of cancer (Non-Patent Literature 2). However, due to limitations of storage and in-vivo delivery of the direct room-temperature plasma, in the present invention, plasma-activated liquid was prepared in the form that can be stored for a long time and effectively delivered into the body.

Specifically, the microplasma jet manufactured in Example 1-1 was equipped with an AC power supply, and gas-phase plasma was jetted onto the surface of 1 mL of DMEM or PBS for 1 to 5 minutes, thereby preparing plasma-activated liquid. As conditions for gas-phase plasma jetting, a voltage of 20 kVp-p and a frequency of 20 kHz were applied, and nitrogen gas was injected at a rate of 10 L/min. When the prepared plasma-activated liquid was directly used as a cell medium, fresh plasma-activated liquid was used, and when the plasma-activated liquid frozen at −20° C. for long-term storage and thawed after a certain period (24 hours to 6 months) was used as a cell medium, low-heat shock plasma-activated liquid was used. In addition, when the fresh plasma-activated liquid was stored in a cold chamber or at room temperature for 1 to 6 months without thawing, plasma stored in a cold chamber or at room temperature was used. In addition, when fresh plasma-activated liquid was stored for 1 to 6 months in a cold chamber or at room temperature without freezing, plasma-activated liquid stored in a freezer or at room temperature was used. Other than DMEM, plasma-activated liquid was prepared using a skin tonic or PBS by the same method as described above, and stored in a cold chamber or at room temperature for 1 to 6 months.

For use as an untreated control with no plasma-activated liquid treatment, only DMEM onto which plasma was not jetted was used as a control. As a control for frozen plasma-activated liquid subjected to low-heat shock, only DMEM was frozen and thawed under the same conditions to be used as a cell medium.

### Example 2

Confirmation of Anticancer Activity of Plasma-Activated Liquid

To confirm that the plasma-activated liquid of the present invention induces the death of cancer cells, a cancer cell killing effect of the plasma-activated liquid of the present invention was compared with that of gas plasma produced by conventional direct jetting.

First, direct gas-phase plasma was jetted onto cancer cells. Specifically, a cervical cancer cell line, i.e., HeLa cells (ATCC CCL-2) was inoculated into 10% FPB-containing DMEM, and cultured for one day. Next day, when the cells were grown to a cell density of 90%, the microplasma jet manufactured in Example 1-1 was prepared at a position 3.8 cm apart from the cells, and jetted gas plasma for 5 minutes, and then the cells were cultured again for 24 hours.

In addition, the cancer cells were treated with the plasma-activated liquid. Specifically, a cervical cancer cell line, i.e., HeLa cells (ATCC CCL-2) was inoculated into 10% FPB-containing DMEM, and cultured for one day. Next day, when the cells were grown to a cell density of 90%, the medium was removed and replaced with the fresh plasma-activated liquid prepared in the DMEM as described in Example 1-3 as a fresh medium, and then the cells were cultured again for 24 hours.

After treatment with gas-phase plasma (direct plasma) or plasma-activated liquid, the HeLa cells cultured for 24 hours were harvested, and double-labeled with 2 μM calcein-AM and 4 μM EthD-1 (Life Technologies) in accordance with a protocol provided by a manufacturer. And then, calcein-A-positive living cells and EthD-1-positive dead cells were observed using a fluorescence microscope (Nikon Inverted Microscope Eclipse Ti-S/L100), and counted. Among the counted cells, by comparing a non-plasma-treated control and a living cell count, relative cell viability (%) was calculated.

As a result, as shown in FIG. 2, compared with non-plasma-treated control, in both of direct plasma-treated HeLa cells and a plasma-activated liquid-treated HeLa cell line, it was confirmed that cell viability was significantly decreased (FIG. 2A), and the plasma-activated liquid can induce the death of HeLa cells, similar to the direct gas-phase plasma, confirming that the plasma-activated liquid has significant anticancer activity (FIG. 2B).

### Example 3

Confirmation of Freeze-Storage Suitability of Plasma-Activated Liquid

<3-1> Confirmation of Anticancer Activity of Plasma-Activated Liquid According to Freeze-Storage

Since it was confirmed that the plasma-activated liquid dissolved in a liquid can kill cancer cells at a level similar to that of the gas-phase plasma which is directly jetted, it was confirmed whether the anticancer activity can be maintained when the plasma-activated liquid is stored for a long time.

Specifically, the plasma-activated liquid prepared using DMEM as described in Example 1-3 was frozen at −20° C. for 24 or 48 hours, thawed and then subjected to low-heat shock, thereby preparing frozen plasma-activated liquid. Afterward, HeLa cells were inoculated into 10% FPB-containing DMEM, and cultured for one day, and when a cell density reached 90%, the medium was removed, and replaced with the prepared fresh plasma-activated liquid (0 hr) or frozen plasma-activated liquid (24 or 48 hrs) as a fresh medium to further culture the cells for 24 hours. After cell culture, relative cell viability (%) was calculated using the method described in Example 2.

As a result, as shown in FIGS. 3A and 3B, it was confirmed that the plasma-activated liquid can exhibit a significant cell killing effect on HeLa cells regardless of freezing, confirming that there was no significant difference in cell killing effect depending on freezing (FIGS. 3A and 3B).

<3-2> Confirmation of Apoptosis Shown in Cancer Cells Treated with Plasma-Activated Liquid

Since it was confirmed that the plasma-activated liquid has a significant cell killing effect on cancer cells, it was confirmed whether such cell killing effect is caused by apoptosis.

Specifically, the plasma-activated liquid prepared using DMEM as described in Example 1-3 was frozen at −20° C. for 1 to 6 months, and thawed to be provided as a medium for the cultured HeLa cells, and the cells were cultured again for 24 hours. Then, the cultured cells were stained with the Alexa 488-binding annexin V antibody (Invitrogen) and propidium iodide (PI; Invitrogen, Eugene, Oreg.), and a cell death type of the dead cells was determined as apoptosis or necrosis using flow cytometry (BD FACSAria III, BD Biosciences, San Jose, Calif.).

As a result, as shown in FIGS. 3C and 3D, it was confirmed that cell death occurred in the plasma-activated liquid-treated cells at a significant level, and the cell death was caused by apoptosis (FIG. 3C). Particularly, it was confirmed that similar levels of cancer cell killing effects are exhibited in both cases of the plasma-activated liquid frozen for 1 month and the plasma-activated liquid frozen for 6 months, respectively (FIG. 3D).

### Example 4

Confirmation of ROS and RNS Levels in Plasma-Activated Liquid

It has been known that the cancer cell killing effect can be exhibited using direct gas-phase plasma, and apoptosis is induced by the generation of ROS and RNA, resulting in the death of cancer cells (Non-Patent Literature 2). Therefore, it was confirmed whether apoptosis of cancer cells can be induced by the generation of ROS and RNS in the plasma-activated liquid of the present invention.

Specifically, the plasma-activated liquid prepared using DMEM in Example 1-3 was frozen at −20° C. for 1 to 6 months and thawed to be used as a medium for the HeLa cells, and then the cells were cultured again for 24 hours. Afterward, the cultured cells were collected, and subjected to an Amplex UltraRed hydrogen peroxide assay (Life Technologies, Invitrogen) and a Griess assay (Life Technologies, Invitrogen) according to protocols provided by the manufacturers, respectively, to measure ROS and RNS levels in the cells.

As a result, as shown in FIGS. 4A and 4B, it was confirmed that hydrogen peroxide and NO are generated in the cells in both cases of the plasma-activated liquid frozen for 1 month and the plasma-activated liquid frozen for 6 months at levels similar to those of ROS and RNS in fresh plasma-activated liquid (FIGS. 4A and 4B). In terms of ROS generation, the concentration of hydrogen peroxide in cells was approximately 37.2 to 40.5 μM in the plasma-activated liquid, confirming that hydrogen peroxide is generated at a considerably increased level, compared with that of the non-plasma-activated liquid-treated control (FIG. 4A). In addition, even when the NO level was identified, it was confirmed that, in both cases of the fresh plasma-activated liquid and the low-heat shock plasma-activated liquid, a significant NO level can be shown (FIG. 4B).

### Example 5

Confirmation of Regeneration Effect of Dermal Cells by Long-Term Stored Plasma-Activated Liquid

<5-1> Confirmation of Regeneration Effect of Dermal Cells by Plasma-Activated Liquid

In order to confirm another use of the plasma-activated liquid, the regeneration effect of the dermal cells by the plasma-activated liquid was confirmed using a wound healing assay.

Specifically, HEK cells were seeded into a 12-well plate containing DMEM and cultured. When the cell density reached 90%, the bottom of the plate having a single layer of cells was scraped to a uniform width using a micro tip, thereby inducing a scratch in the cells. Afterward, the medium was replaced with fresh plasma-activated liquid prepared using the DMEM as described in Example 1-3 to further culture the cells for a total of 24 hours. Immediately and 12 hours after cell culture, the cells were observed using a phase difference microscope to confirm whether the interval between wounds caused on the bottom of the plate was decreased.

As a result, as shown in FIG. 5, compared with a control cultured in DMEM, in which the distance between wounds is not significantly decreased, it was confirmed that, the HEK cells cultured in the fresh plasma-activated liquid are proliferated and thus the distance between the wounds was decreased (FIG. 5).

<5-2> Confirmation of Dermal Cell Regeneration Effect by Plasma-Activated Liquid Stored in Cold Room or at Room Temperature

Since it was confirmed that the wounds of the dermal cells can be regenerated by the fresh plasma-activated liquid, it was confirmed whether such effect can be maintained even in the stored plasma-activated liquid.

Specifically, wounds of the cells were caused by the method described in Example 5-1, the plasma-activated liquid prepared in DMEM as described in Example 1-3 and stored in a cold chamber or at room temperature for 6 months was used as a cell medium to further culture the cells for a total of 72 hours. One minute and 24 hours after cell culture, the cells were observed using a phase difference microscope to confirm whether the distance between the wounds occurring on the plate bottom was decreased. Other than the plasma-activated liquid prepared using DMEM, it was confirmed that a wound healing effect was shown even in the plasma-activated liquid prepared using a skin tonic or PBS and stored in a cold chamber for 6 months by the same method as described above.

As a result, as shown in FIGS. 6 and 7, it was confirmed that an effect of regenerating dermal cells was maintained in the plasma-activated liquid stored in a cold chamber or at room temperature for 6 months, resulting in a significant wound healing effect, which is significantly increased as compared with direct jetting of the gas-phase plasma (FIG. 6). In addition, other than the plasma-activated liquid using DMEM, the plasma-activated liquid prepared using a skin tonic or PBS also exhibits a significant effect of regenerating dermal cells due to a decrease in distance between wounds even after long-term storage for 6 months (FIG. 7).

### Example 6

Confirmation Whether Long-Term Stored Plasma-Activated Liquid is Associated with Collagen and Melanin Synthesis

Since it was confirmed that the plasma-activated liquid of the present invention can still maintain a skin regeneration effect even when stored for a long time, a change in melanin and collagen synthesis according to treatment with plasma-activated liquid was confirmed.

Specifically, the plasma-activated liquid prepared using PBS as described in Example 1-3 was stored in a cold chamber or at room temperature for 6 months. Afterward, a malignant skin cancer cell line, i.e., B16 melanoma cells, was inoculated into a 10% FPB-containing DMEM, and cultured for one day. When a cell density reached 90%, the medium was removed, and replaced with the plasma-activated liquid stored in a cold chamber or at room temperature to further culture the cells for 48 hours. After cell culture, the cells were harvested to quantify the intracellular collagen concentration using a hydroxyproline assay kit, and quantify the concentration of a melanin pigment using the method suggested by Hosoi et al. (Hosoi, J., Abe, E., Suda, T. and Kuroki, T. Regulation of melanin synthesis of B16 mouse melanoma cells by 1 alpha, 25-dihydroxyvitamin D3 and retinoic acid. Cancer Res. 45, 1474 (1985)). The quantified concentrations of the collagen and the melanin pigment were calculated as relative concentrations based on the control cultured in PBS in which plasma-activated liquid was not contained.

As a result, as shown in FIG. 8, it was confirmed that the plasma-activated liquid stored in a cold chamber and plasma-activated liquid stored at room temperature for 6 months were decreased in melanin synthesis, thereby having a whitening effect, and were increased in collagen synthesis, thereby having a skin regeneration effect (FIG. 8).

1-26. (canceled)

27. A composition comprising a plasma-activated liquid as an active ingredient.

28. The composition of claim 27, wherein the plasma-activated liquid is prepared by jetting plasma generated in an atmospheric-pressure plasma jet onto an ion-containing solution.

29. The composition of claim 28, wherein the ion-containing solution is selected from the group consisting of water, a skin tonic, a culture medium, a buffer solution, and a combination thereof.

30. The composition of claim 27, wherein the plasma-activated liquid maintain its activity after being stored in a freezer at −20 to 0° C.; in a cold chamber at 0.1 to 10° C.; or at room temperature at 10 to 40° C.

31. The composition of claim 30, wherein the storage is maintained for 1 day to 6 months.

32. The composition of claim 27, which is a cosmetic composition, a pharmaceutical composition, a dietary supplement, or a health functional food.

33. The composition of claim 32, wherein the cosmetic composition has a skin-whitening activity.

34. The composition of claim 32, wherein the pharmaceutical composition has a skin regeneration and/or wound healing activities.

35. The composition of claim 32, wherein the pharmaceutical composition has a cancer prevention and/or cancer treatment activities.

36. The composition of claim 32, wherein the health functional food has a cancer prevention and/or cancer alleviation activities.

37. A method selected from the group consisting of following (a)-(d):
(a) a method for whitening a skin of a subject in need thereof, comprising administering an effective amount of a plasma-activated liquid to the subject;
(b) a method for skin regeneration and/or wound healing of a subject in need thereof, comprising administering an effective amount of a plasma-activated liquid to the subject;
(c) a method for preventing and/or treating a cancer in a subject in need thereof, comprising administering an effective amount of a plasma-activated liquid to the subject; and
(d) a method for preventing and/or alleviating a cancer in a subject in need thereof, comprising administering an effective amount of a plasma-activated liquid to the subject.

38. The method of claim 37, wherein the plasma-activated liquid of (a)-(d) is prepared by jetting plasma generated in an atmospheric-pressure plasma jet onto an ion-containing solution.

39. The method of claim 38, wherein the ion-containing solution is selected from the group consisting of water, a skin tonic, a culture medium, a buffer solution, and a combination thereof.

40. The method of claim 37, wherein the plasma-activated liquid of (a)-(d) maintain its activity after being stored in a freezer at −20 to 0° C.; in a cold chamber at 0.1 to 10° C.; or at room temperature at 10 to 40° C.

41. The method of claim 40, wherein the storage is maintained for 1 day to 6 months.

42. The method of claim 37, wherein the plasma-activated liquid of (a) is in a cosmetic composition form and is applied to the skin of the subject.

43. The method of claim 37, wherein the plasma-activated liquid of (b) is in a pharmaceutical composition form and is administered orally or non-orally to the subject.

44. The method of claim 37, wherein the plasma-activated liquid of (c) is in a pharmaceutical composition form and is administered orally or non-orally to the subject.

45. The method of claim 37, wherein the plasma-activated liquid of (d) is in a dietary supplement form or a health functional food form and is administered orally to the subject.
